(19) United States
(12) Patent Application Publication
(10) Pub. No.: US 2023/0366671 A1
(43) Pub. Date: Nov. 16, 2023

(54) FIELD SCANNING OPTICAL COHERENCE TOMOGRAPHY

(71) Applicant: Miami University, Oxford, OH (US)

(72) Inventor: Hui Wang, Oxford, OH (US)

(21) Appl. No.: 18/134,929

(22) Filed: Apr. 14, 2023

Related U.S. Application Data
(60) Provisional application No. 63/330,999, filed on Apr. 14, 2022.

(51) Int. Cl. G01B 9/02091; A61B 3/10; A61B 3/00; A61B 5/00

(52) U.S. Cl. CPC G01B 9/02091; A61B 3/102; A61B 3/0025; A61B 5/0066

(57) ABSTRACT

A Field Scanning OCT (FSOCT) system that overcomes the bottleneck of imaging speed through simultaneous (parallel) detection of photons from a sample. This provides phase stability during imaging. The herein-disclosed FSOCT methods and devices detect backscattered photons in parallel simultaneously from multiple locations without relying on mechanical motion to capture them at offset positions at different times. This significantly improves the performance of OCT imaging.

### REFERENCE TO RELATED APPLICATIONS

This application claims the benefit of U.S. Provisional Application No. 63/330,999 filed Apr. 14, 2022.

### BACKGROUND OF THE INVENTION

This invention relates generally to optical coherence tomography methods and devices used to measure the characteristics of human tissue and other samples.

Optical coherence tomography (OCT) has emerged as an important imaging modality used in several clinics, especially in ophthalmology and dermatology. Acquiring deep-tissue images at cellular resolution is highly desirable for both biological research and clinical diagnosis. However, tissue heterogeneity can introduce optical aberrations, which then degrade the lateral point spread function (PSF), or lateral resolution. For example, imperfect ocular optics is common during retinal imaging, as is the skull during brain imaging. Adaptive optics OCT (AO-OCT) addresses this challenge by reshaping the wavefront of the illumination beam to focus the beam to diffraction-limited PSF in a targeted region. Wavefront sensor-based AO-OCT (WAO-OCT) optimizes PSF based on the metric from a wavefront sensor. Despite the success of WAO-OCT demonstrated in research labs, translating WAO-OCT into clinics has been hampered by complexity, cost, and size.

As an alternative, simpler sensorless AO-OCT (SAO-OCT) eliminates the wavefront sensor by using image metrics (i.e., intensity or sharpness) to optimize PSF. However, since image metrics are only indirectly related to PSF, SAO-OCT cannot guarantee optimal results globally. In addition, because a strong and stable backscattered signal is required during SAO-OCT optimization iterations, the image metric acquired through 2D enface scanning is susceptible to motion. The slow iteration has hindered the clinical adaptation of SAO-OCT.

Recently, artificial neuron networks (ANNs) have been explored to derive the aberrated wavefront using the PSF generated from a point source. Trained ANNs can optimize the wavefront instantly, eliminating time-consuming iteration. To accomplish this, the ANNs must be trained with a universal metric, like PSF, or its counterpart in the frequency domain, modulated transfer function (MTF). If an image metric is used, the ANN has to be retrained when the imaged object or the system optics are different. Retraining is not acceptable during clinical diagnosis as it is costly and time-consuming.

It appears that the ideal metric for SAO-OCT should be either PSF or MTF. However, accessing the PSF or MTF in a scattering medium without a guide star is challenging. To the applicant's knowledge, no solution has been discovered.

The contrast in OCT images originates from backscattered photons, resulting from the refractive index variation in tissue. New contrasts, such as tissue property-related optical attenuation coefficient (OAC), have been intensively studied to improve the diagnostic capability of OCT. For instance, a decrease of the OAC in the retinal nerve fiber layer has been linked to glaucoma severity. In dermatology, OAC has been tested to monitor the healing process of burn wounds.

OAC is derived from the original scattering contrast and depends on the backscattered photons detected by OCT. The backscattered photons consist of least scattered photons (LSPs) and multiple scattered photons (MSPs). LSPs “remember” the spatial locations to which they were backscattered, inasmuch as the locations can be surmised from the data measured. MSPs “lose” this memory, inasmuch as the locations cannot generally be surmised from the data measured. Although OCT uses a low-coherent gate to capture LSPs and reject MSPs, certain MSPs can still enter the coherent gate and skew the quantification of OAC.

Currently, the extraction of tissue-related OAC is largely based on the single-scattering model, which has two major limitations. First, the single-scattering model ignores MSPs, which is problematic when quantifying the OAC of highly scattering media, such as the skin or in deep tissue, where MSPs are dominant. Second, multiparameter nonlinear fitting introduces significant variation. A minimum of three parameters—focal depth (zf), Rayleigh range (zR), and OAC (μs)—are required to fit a nonlinear equation and are also interdependent. Prior knowledge about zf and zR is required to minimize uncertainty during the fitting process, but it can only be obtained by carefully controlling the imaging process. Despite these efforts, significant variation in OAC can still be observed and the underlying mechanism of this variation has not yet been well understood. Translating OAC measurement into clinical use faces challenges due to patients' different ocular optics and motion artifacts during in vivo imaging.

In conventional OCT, the illumination and detection beams share the same optical path, while in BO-OCT, as shown in FIG. 1, the detection beam is offset from the illumination beam by a small distance Ar. In applicant's published paper (E. Bo, L. Wang, J. Xie, X. Ge and L. Liu, “Pixel-Reassigned Spectral-Domain Optical Coherence Tomography,” in IEEE Photonics Journal, vol. 10, no. 2, pp. 1-8, April 2018, Art no. 3900408, doi: 10.1109/JPHOT.2018.2813523), the OCT images from the offset positions were used to improve the lateral resolution. In a previous patent application (U.S. Pat. No. 10,942,023, incorporated herein by reference), the ability to acquire images at a depth deeper than the depth of conventional OCT is disclosed. FIG. 1 is an illustration of the notation and relation of optics in BO-OCT. The illumination beam is focused by a lens and photons are backscattered due to refractive index variation R(\vec{r}, z). The detection beam may capture backscattered photons at an offset position from the illumination beam.

The setup illustrated in prior art systems shown in FIGS. 2 and 3 is alleged to capture the offset photon. The lens L2 can be shifted or movable, and the photons along the path 32 can be captured to form an OCT image. With the prior art apparatus shown in FIG. 4, the detection fiber was offset by a stepper so that the photon from an offset position could be captured. From the hardware, both schemes are similar. The lens or the fiber must be moved step-by-step over a period of time to capture photons from multiple offset positions. Such motion can be effected manually or automatically. However, mechanical motion limits the imaging speed. In applications, it is essential to capture images in real-time, such as above 20 frames per second. The offset based on the mechanical motion will eventually become the bottleneck of the technology. In addition, the phase stability of the OCT signal is lost due to the motion.

### SUMMARY OF THE INVENTION

Disclosed herein is an interferometer using methods to capture backscattered photons from at least one position that has a small offset from the illuminated area. Detection of photons from an offset position is known, and the technology has been used in other optical detection schemes, such as Raman scattering and fluorescence detection. However, the disclosed technology of parallelly detecting photons at an offset position and processing the data are different, significantly affecting how the technology can be realized and used in clinics. Furthermore, the devices and methods disclosed are different from the prior art as noted herein.

It is demonstrated herein that the offset OCT images can be used to reconstruct a Backscattered Photon Profile (BSPP). A BSPP permits visualization of the beam profile in the scattering medium, and an example of this is shown in FIG. 6. With a BSPP, one can track the focus during imaging, quantifying the distribution of LSPs and MSPs, using point spread function (PSF) or modulated transfer function (MTF) as the feedback for adaptive optical imaging, acquiring stable phase during imaging to realize digital refocusing.

Because the skirt of the BSPP represents MSPs, the skirt can be ignored and one can use only the LSPs. Thus, two things may be accomplished with BO-OCT. One can (a) obtain the PSF and MTF, and (b) separate the MSPs from the LSPs. But the problem still remains that it takes too long to gather data, such as 100 images, needed to have a reliable BO-OCT reading. The mechanical translation for offsetting the detection beam in prior art OCT systems can limit imaging speed, significantly affecting clinical use. In addition, because the prior art acquired offset images sequentially, the phase stability was lost. Patients cannot hold their eyes still for even a few seconds, but for a reliable reading the data must be gathered in real-time, such as at about 30 images per second.

The present invention relates to a Field Scanning OCT (FSOCT) system, which overcomes the bottleneck of imaging speed. The FSOCT system accomplishes this through simultaneous (parallel) detection and thus provides both speed and phase stability during imaging. This thereby provides a solution to the limitations of previous methods and devices.

The herein-disclosed FSOCT methods and devices overcome these problems by detecting backscattered photons in parallel simultaneously from multiple orientations and/or locations, without relying on mechanical motion to capture them at offset positions. For example, with the invention 100 OCT images may be obtained at the same time over a 100 micron space. This significantly improves the performance of OCT imaging by enabling faster and more stable imaging for clinical use.

The disclosed technology is described herein inasmuch as the equipment described can simultaneously detect offset photons, which means photons from parallel positions. Furthermore, the disclosed technology is described herein with methods for different innovative applications.

### BRIEF DESCRIPTION OF THE DRAWINGS

FIG. 1 is a schematic illustrating the notation and relation of optics in prior art BO-OCT.

FIG. 2 is a schematic side view illustrating a prior art setup used to capture backscattered photons.

FIG. 3 is a schematic end view illustrating the prior art setup of FIG. 2.

FIG. 4 is a schematic side view illustrating another prior art setup used to capture backscattered photons.

FIG. 5 is a schematic end view illustrating the prior art setup of FIG. 4.

FIG. 6 is a group of backscattered photon profiles gathered according to the invention at different focal positions.

FIG. 7 is a group of graphs illustrating the quantification of PSF auto correlation functions, MTFs, and beam waist.

FIG. 8 is a schematic view illustrating a field scanning OCT (FSOCT) with spectrometers.

FIG. 9 is a flow chart illustrating steps in a method of extracting true PSF from the PSF autocorrelation function by controlling reference numerals 20 and 21 of FIG. 8.

FIG. 10 is a schematic view illustrating a field scanning OCT with a swept light source.

FIG. 10B is an alternative optical layout to the embodiment shown in FIG. 10.

FIG. 11 is a schematic view illustrating field scanning optical coherence microscopy (FSOCM).

FIG. 11B is an alternative optical layout to a portion of the embodiment shown in FIG. 11.

FIG. 12 is a flow chart illustrating data and image processing steps.

FIG. 13 is a schematic illustrating the steps of reconstructing BSPP with neighbor A-scans.

FIG. 14 is a schematic illustrating the FSOCT illumination beam 1 focused on the tissue 4 through a lens 3 for focus locking.

FIG. 15 is a schematic illustrating phase variation at two different times, T1 and T2 on moving particles.

FIG. 16 is a schematic illustrating FSOCT guided adaptive optical imaging in which a wavefront shaping component 3, which may be a deformable mirror, can be employed to modify the wavefront of the illumination beam that is focused onto a sample 7 through a lens or lenses 6.

FIG. 17 is a schematic illustrating a flowchart of neuron network training for adaptive imaging.

FIG. 18 is a schematic illustrating a flow chart of extracting the attenuation coefficient and anisotropy.

FIG. 19 is a schematic illustrating an alternative embodiment of the present invention.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

Patent application Ser. No. 63/330,999, which is the above-claimed priority application, is incorporated in this application by reference.

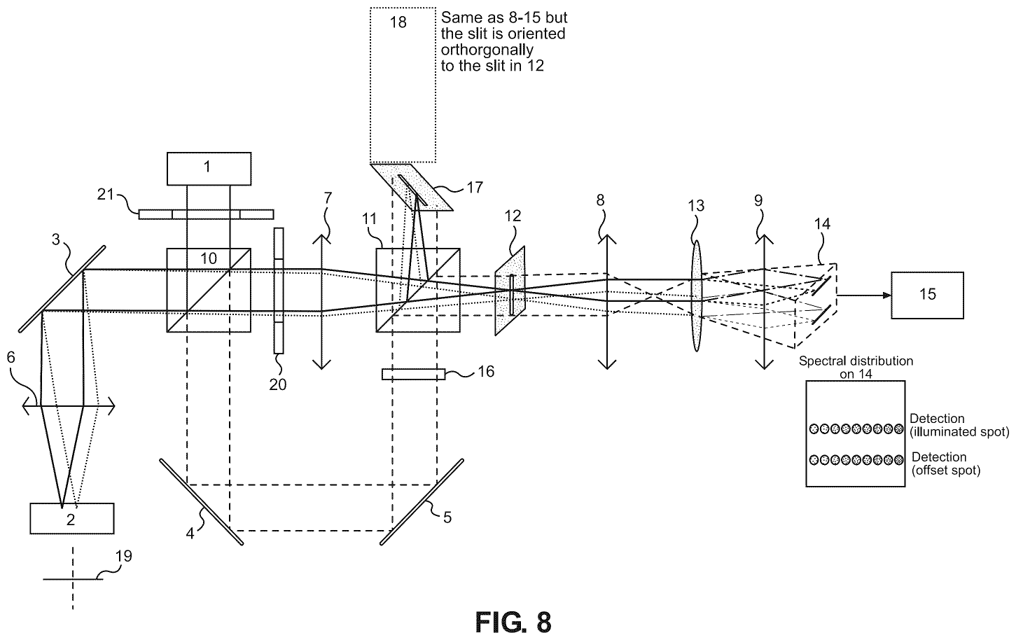

Looking first to an embodiment shown in FIG. 8, the disclosed system includes a broadband light source 1 that emits light in a broad range of wavelengths. It is contemplated that the light may be in a wide portion of the light spectrum, and may be, for example from 400 nanometers to 2.5 microns in wavelength. The light is split by a beam splitter 10 to a scanner 3 and focused by a lens 6 on a sample 2. The backscattered photons from the sample 2 are detected in parallel by a detector array 14, which may comprise a photodetector array or charge-coupled device (CCD) with M rows and N columns of photodetectors. A detector array has a plurality of photodetectors that are closely packed together, such as on a charge-coupled device. It should be noted that a detector array need not be an integrated circuit containing an array of linked, or coupled, capacitors as with a CCD. Nevertheless, the photodetectors in a detector array are linked so that they may detect light simultaneously as the light falls on the detector array. This permits a photodetector array to detect light backscattered from a sample, including light backscattered from an illumination point and many offset points.

The detector array 14 detects spectral interference fringes. The light backscattered from the sample 2 (the solid line) is focused on a slit 12 after a focusing lens 7. The slit 12 serves as a spatial filter to gather the signal from only one orientation, such as the solid line 19, and filter the signal from other orientations of the sample 2. The signal from different offset positions on the sample 2 is first dispersed by a dispersive component (i.e., grating 13), then focused on different rows of the detector array 14 so data can be collected at the detector array 14 and stored in a data storage or further processed by a computer 15. After the slit 12, the light is collimated again by a lens 8 and then dispersed by a grating 13 and focused onto the detector array 14 by a lens 9. In order to obtain further enhanced precision, one can add a second spatial filter, such as the slit 17, along with duplicates of the components that follow slit 12 (all such duplicate components are shown in FIG. 8 as reference numeral 18) to filter the offset signal from other orientations on the sample 2, such as the dashed line 19′. Another detector array may receive the beam after a dispersive component disperses the signal that passes through the slit 17.

The dashed line following the light path reflected by mirrors 4 and 5 is the reference light path, which will illuminate the detector array and interfere with the photons backscattered from the sample. One can further reshape the reference light into a line using optical components 16 like a cylinder lens, a Powell lens or other phase modulators can also be used to further reshape the reference light to generate a uniform reference light field on the photodetector array(s) 14 and those in reference numeral 18.

The spectral interference fringes will be acquired by the detector array 14 for data processing, which may be by a computer, or storage in a computer's local drive. Only the light paths of two positions, the illuminated spot and a single beam offset, are shown in FIG. 8. However, in practice, many offset positions can be simultaneously detected and the number is determined by the number of rows M of the detector array 14 and will typically be in the dozens or hundreds of offset positions. The solid black line representing the light path of the backscattered light from the illuminated spot may be dispersed based on wavelength to the central column of the array 14. The backscattered photons from the offset position illustrated as the dotted line may be dispersed to the lower row of the detector array 14. As the array detector 14 has M columns, the spectra from M offset positions can be simultaneously detected. The reference light path, indicated by the dashed line following the light path reflected by mirrors 4 and 5, illuminates the detector array 14 and interferes with the light backscattered from the sample 2. The spectral interference fringes are then acquired for data processing. As noted above, a similar optical path from 12 to 14 can be built along the path including the slit 17 and duplicate components from the path from 12 to 14 represented by reference numeral 18, except the grating should be aligned with the slit so that the light from the slit can be dispersed in a parallel manner on the CCD. A difference between the two paths is that the orientation of the slit 17 is aligned with the backscattered photons along the dashed line of 19′. The slit 17 is preferably transverse, and more preferably perpendicular, to the slit 12, just as the dashed line 19′ is perpendicular to the solid line 19. Offset positions at other orientations can be detected by changing the orientation of one or both of the slits 12 and 17.

In addition to configuring the setup with free-space components, fiber components can also be used as an alternative for the same purposes. It is also possible to detect the backscattered photons from more than two orientations by further splitting the backscattered light with multiple beam splitters dispersive components and lenses after the beam splitter 11. The configuration is the same for adapting slits with different orientations.

The data processing flow after acquiring the images with FSOCT or FSOCM is described below. In either spectrometer-based OCT or swept light source-based OCT, the processing of an A-scan of OCT images follows a standard process typically including DC removal, resampling, dispersion compensation, Fourier transform, and image reconstruction. Different and specific data analyses based on FSOCT are described in detail, and the fact that the offset OCT images can be used to reconstruct a BSPP is demonstrated. The BSPP allows one to visualize the beam profile in the scattering medium. With BSPP, one can track the focus during imaging, quantifying LSPs and MSPs, using point spread function (PSF) or modulated transfer function (MTF) as the feedback for adaptive optical imaging, and highly sensitive phase measurement, as described below.

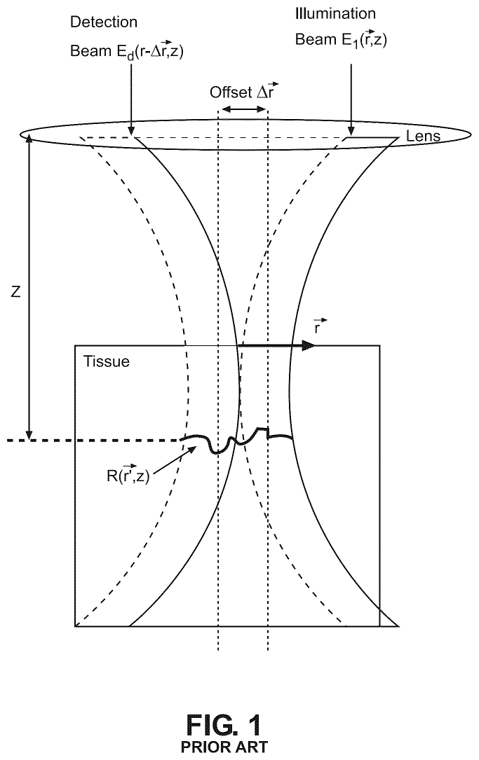

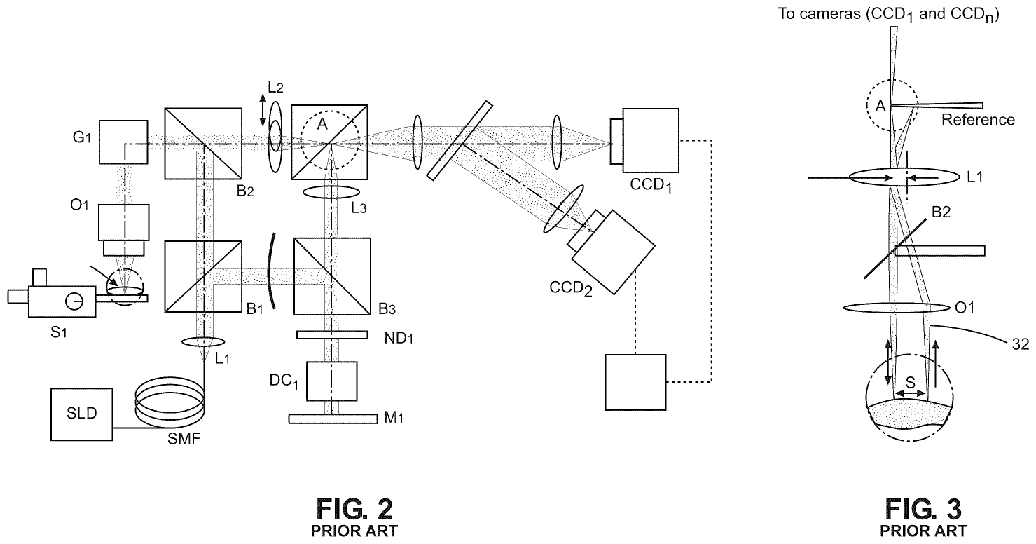

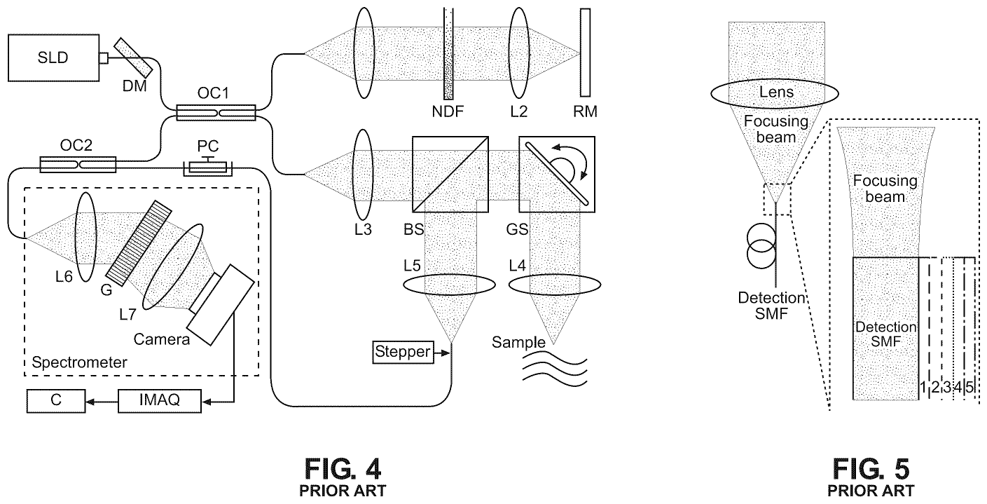

As shown in FIG. 1, the detection beam of a conventional, prior art system is offset from the illumination beam by a small distance Δr. Considering cylindrical coordinates, R(\vec{r}, z) is the scattering potential determined by refractive index variation in a scattering medium. From the first-order Born approximation, the light field coupled back into the detection beam at an offset Δ\vec{r}, Det(Δ\vec{r}, z), can be calculated based on the mode match between the scattered field, Eᵢ(\vec{r}, z)R(\vec{r}, z), and the offset detection field, E_{d}(\vec{r}−Δ\vec{r}, z). Because the illumination beam field and the detection beam field are generated by the same lens except for a small offset, it is reasonable to assume E*_{d}(\vec{r}−Δ\vec{r}, z)=Eᵢ(\vec{r}−Δ\vec{r}, z), if the aberration due to the offset is neglected. Then

Det(Δ\vec{r},z)∝∫Eᵢ(\vec{e}−Δ\vec{r},z)Eᵢ(\vec{r}z)R(\vec{r},z)d²\vec{r} (1)

The light intensity distribution, I_{d}(Δ\vec{r},z), measured by BO-OCT can be written as

I_{d}(Δ\vec{r},z)∝|∫Eᵢ(\vec{r}−Δ\vec{r},z)Eᵢ(\vec{r},z)R(\vec{r},z)d²\vec{r}|² (2)

Multiple A-scans can be averaged by scanning the illumination beam in a small range to reduce speckles. Assuming R(\vec{r},z) is independent and random at different locations, the averaged intensity can be simplified as

I(Δ\vec{r},z)∝∫Hᵢ(\vec{r}−Δ\vec{r},z)Hᵢ(\vec{r},z)d²\vec{r}=Hᵢ(\vec{r},z)*Hᵢ(−\vec{r},z)=Hᵢ(\vec{r},z)*Hᵢ(\vec{r},z) (3)

Here, H(\vec{r},z)=|Eᵢ(\vec{r}₁,z₁)|², which is called intensity point spread function or just PSF, a real function. The symbol * is used to represent correlation and the symbol * is used to represent convolution. Fourier transform can be conducted on both sides of Eq. (3) relative to \vec{r}, then

[symbol]

[I(Δ\vec{r},z)]∝ [symbol] [Hᵢ(Δ\vec{r},z)] [symbol] [Hᵢ(−Δ\vec{r},z)]=|M(fᵣ,z)|² (4)

Here, M(fᵣ,z) represents MTF. Therefore, the averaged BO-OCT intensity signal is the PSF autocorrelation or the inverse Fourier transform of the squared MTF at different depths. Thus, with BO-OCT, one can reconstruct the depth-resolved PSF autocorrelation or MTF, two parameters that are widely used for evaluating imaging system performance but have never been achieved as depth-resolved forms in a scattering medium.

The illumination beam and the detection beam could have different apertures or wavefronts.

Note that I is the cross correlation between the PSFs of the illumination beam and the detection beam. As the two PSFs are different, I is not a symmetric function. The asymmetric aberration such as a comatose state can be detected in this way.

If considering a Gaussian approximated illumination beam and only LSPs, the normalized detected intensity at each depth with BO-OCT can be written as

$$I ( \vec{Δ r} , z ) = {e}^{- \frac{Δ {r}^{2}}{{w}_{2}^{2}}} \qquad ( 5 )$$

where

$${w}_{z}^{2} = {w}_{0}^{2} [ 1 + {( \frac{z - d}{{z}_{R}} )}^{2} ] ,$$

z_{R} is the Rayleigh range of the Gaussian beam, d is the distance from the surface of an imaged medium to the beam focus, and w₀ is the beam waist at the focus. From Eq. (5), the reconstructed LSPs profile in the scattering medium is the illumination beam field, showing the beam waist at different depths, the focal position, and the Rayleigh range. Therefore, even if the wavelength and the optics used to focus the illumination beam are not known, information about how the light beam is distributed in the scattering medium based on the reconstructed LSPs profile can be obtained with FSOCT.

In FSOCT, the depth-resolved PSF autocorrelation function can be accessed, and then this function can be used to obtain the true PSF. To control the PSFs of the illumination and detection beams, components 20 and 21 in FIG. 8 can be used, and these may be apertures or wavefront controllers. The equation (3) can be modified as

I(Δ\vec{r},z)=Hᵢ(\vec{r},z)*H_{d}(\vec{r},z) (6)

where I is the cross-correlation between the PSFs of the illumination beam and the detection beam. In an optical system, the PSF of a beam with a large diameter suffers significant distortion due to the aberration of the optical system, while the distortion is small with a beam having a small diameter.

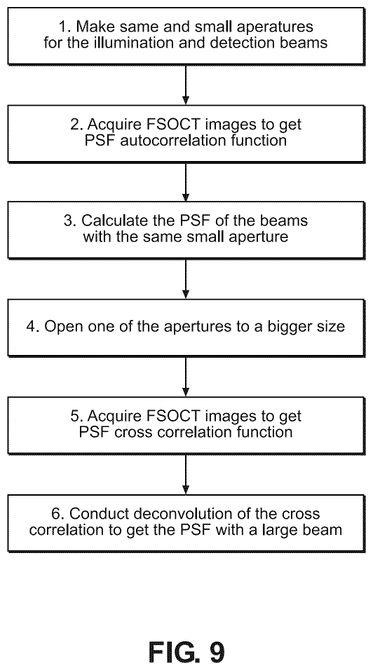

The following steps, which are illustrated in FIG. 9, show the method of extracting true PSF from the PSF autocorrelation function by controlling components 20 and 21 shown in the embodiment of FIG. 8. In step 1, assuming the beam size is small enough, the PSFs of the reference and sample arms can be approximated to a Gaussian distribution and free from aberration. In step 2, the FSOCT images are obtained as described above in relation to FIG. 8. From equation 5, the PSF can be determined in step 3. In step 4, one aperture, such as that of the illumination arm, is opened to a larger size, causing the PSF of the illumination beam to become distorted due to the aberration of the optical system. In step 5, the PSF autocorrelation function obtained through FSOCT can be described by Eq. (6) because the apertures of the illumination beam and the detection beam are different. In step 6, deconvolution can be used to calculate the PSF of the larger beam, as the PSF of the smaller aperture was obtained in step 3.

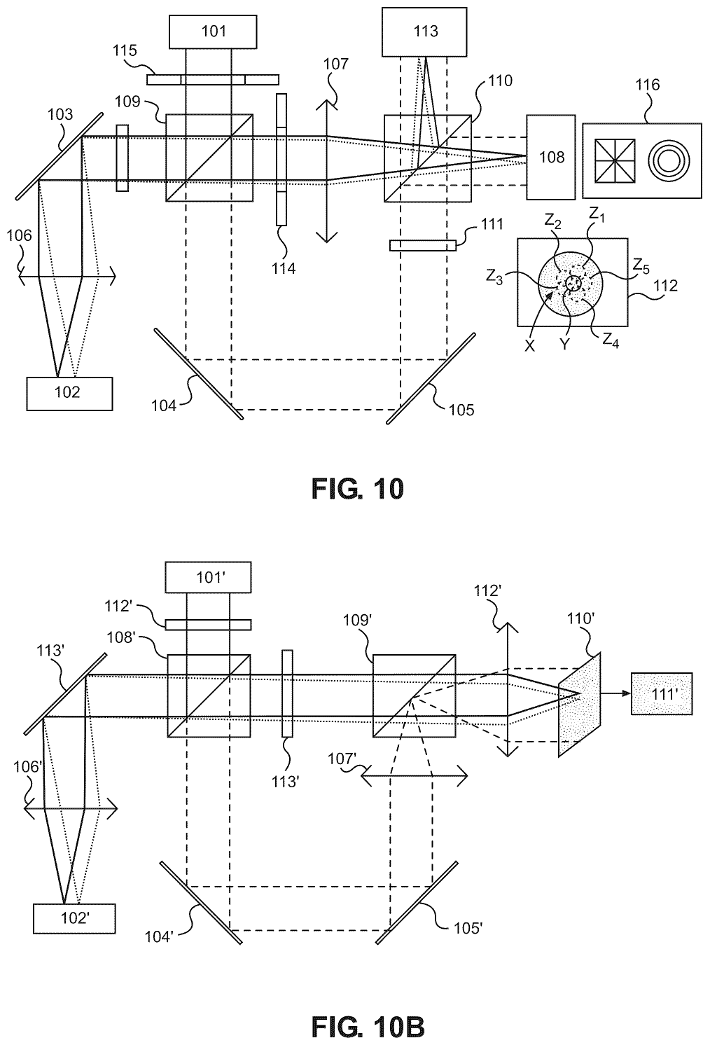

As shown in FIG. 10, which is an alternative embodiment, a swept light source 101 may be used instead of the broadband light source and the dispersion component (i.e., grating 13) shown in FIG. 8. This allows a detection array 110 to be placed directly after the beam splitter 109, without the need for a dispersion component required in the FIG. 8 embodiment. The backscattered photons from the sample 102 are focused through the lens 107 onto the detection array 110, enabling the simultaneous acquisition of OCT images from all around the illumination spot, as shown in pattern 112 in FIG. 10. These images may be stored in the storage 111 or immediately processed by a computer. The gray area X in pattern 12 represents the reference beam, the solid center circle Y represents the beam formed by the solid light path, and the dashed line circles Z₁, Z₂, Z₃, Z₄, and Z₅ indicate the light from some of the many offset positions relative to the center circle Y. With this setup, one can capture the backscattered photons from offset positions (Z₁-Z₅) around the illumination beam. Although only five offset positions are shown in FIG. 10, the number of captured offset positions is determined by the number of pixels in the detector array 10.

In addition, a second detection array 113 can detect a similar pattern with an opposite phase, forming balanced detection and reducing image noise after subtracting the signal acquired with arrays 108 and 113. Both detection arrays 108 and 113 are similar to CCDs and may have M rows and N columns of pixels. However, having a larger number of pixels increases the amount of data that needs to be processed by subsequent units such as a computer. To address this issue, a detector array can be designed in various configurations to reduce the data processing load. For example, for the first pattern in the box 116 of FIG. 10, only four lines of pixels may be required to be analyzed. These could be in the form of a cross or a circle. Alternatively, pixels falling on two or more concentric circles may be analyzed, as may other sampling shapes. The reference arm introduces a uniform illumination on a photodetector array, like a CCD or a CMOS. The photons backscattered from the sample will be projected on the detector array 108 and 113.

There are different variations of this setup. Reference numerals 114 and 115 in FIG. 10 are components which can be used to create different wavefronts between the illumination beam and the detection beam. The illustration of FIG. 10B is an alternative optical layout. However, for realizing FSOCT, the most critical part is to be able to capture the backscattering photons with a photodetector array in 2D simultaneously. Reference numerals 112′ and 113′ in FIG. 10B are components which can be used to create different wavefronts between the illumination beam and the detection beam.

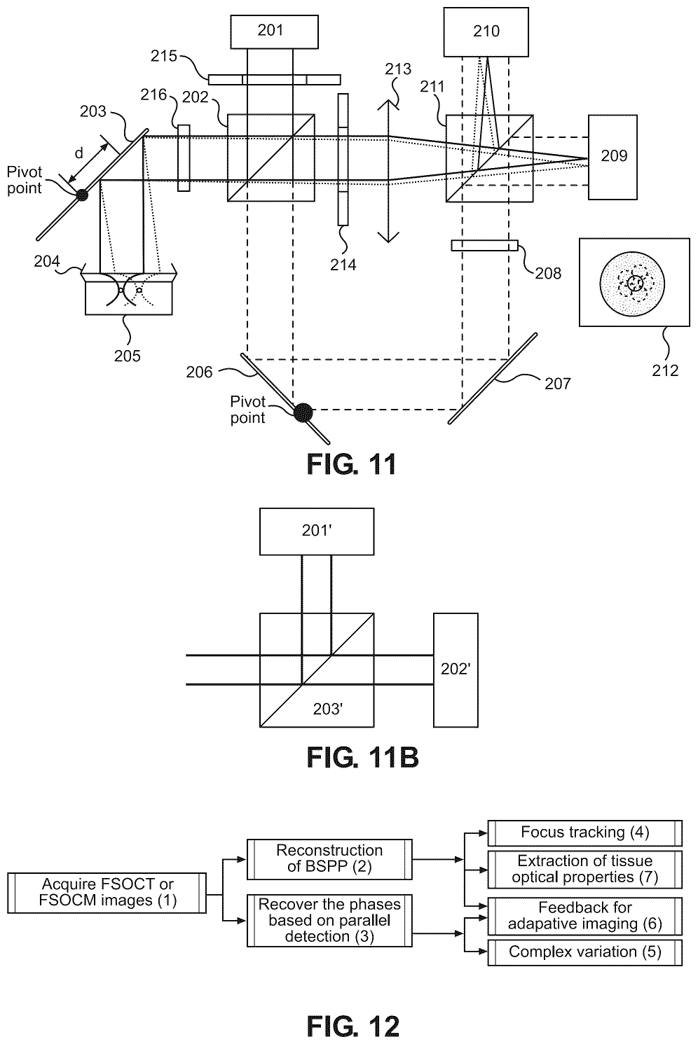

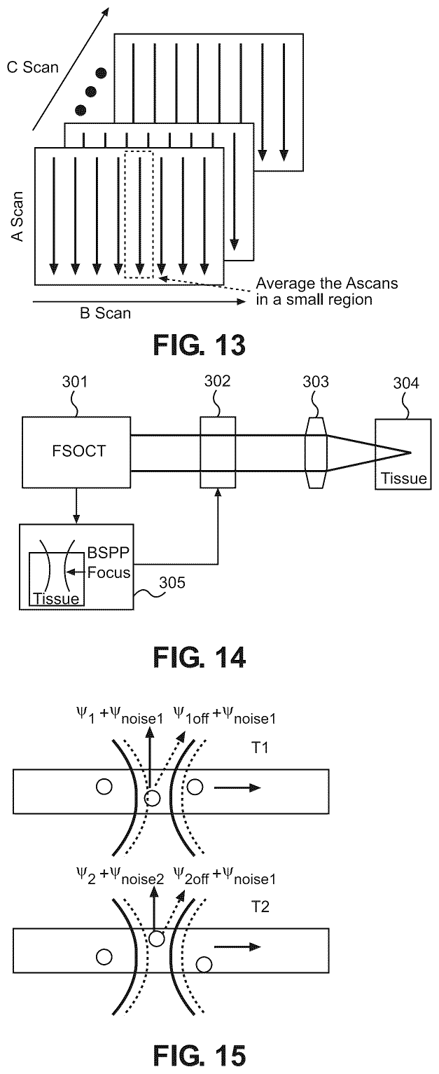

Optical Coherence Microscopy (OCM) is a variation of OCT that captures an enface view image at a specific depth in a sample. Similarly, FSOCM is an improvement OCM just as FSOCT is an improvement of OCT. In FSOCM, an example of which is shown in FIG. 11, a high numerical aperture lens 206 is used to capture backscattered photons from the focal spot and the offset positions. The light source 201 is a broadband light source that is split into the scanner or scanning mirror 203 and then focused through the lens 204 to the sample 205. To generate an interference pattern or phase variation across the detector arrays 209 and 210, a phase modulation must be introduced during scanning. The phase modulation can be introduced in either the sample arm or the reference arm.

There are different ways of generating such phase change, and they can all be adapted to FSOCM. In one example, the light beam illuminating the sample 205 can be shifted from the pivot point of the scanner 203. During scanning, a phase modulation will be introduced. The photodetector arrays 209 and 210 can capture such phase modulation, and the signal will be demodulated during data processing. Components 214 and 215 can be used to create different wavefronts or apertures between the illumination beam and the detection beam. Another way to generate phase modulation is to introduce a configuration in the reference arm that is synchronized with the scanning mirror. For example, a scanner can replace the mirror 206 with a beam offset from the pivot point. This scanner must be synchronized with the scanner 203. Alternatively, a phase modulator 216 or 208 can be introduced in the sample or reference arm and synchronized with the scanning mirror.

The backscattered photons from the illuminated and surrounding spots are simultaneously focused on the detector arrays 209 and 210 through the lens 213. The data from the detector arrays 209 and 210 can be captured, stored and/or processed. As the phase modulation is introduced during scanning, interference modulation due to the phase modulation is captured in the form of amplitude modulation (AM). The AM signal from each pixel can be demodulated using Fourier transform or the principle of a locked-in amplifier or filters to recover the interference signal and reconstruct the FSOCM image at a specific depth.

Reference for the modulation method described in (a): https://doi.org/10.1117/1.3155523.

Reference of OCM: https://link.springer.com/protocol/10.1007/978-1-4939-6810-7_12.

Another embodiment is referred to as a fiber bundle-based OCT or OCM (FSOCT or FSOCM, respectively). The embodiment of FIG. 19 shows a novel apparatus for a method of imaging human organs through an endoscope by using a fiber bundle. The apparatus can be used for FSOCT and FSOCM.

The invention uses a fiber bundle which provides flexibility for imaging in a cavity. FIG. 19 shows an FSOCT/FSOCM based on fiber components. A light source 503, which could be a broadband light or swept light source, is coupled into a fiber coupler 504. The outputs of the fiber coupler 504 are the inputs of the fiber couplers 505 and 507. The fiber couplers 505 and 507 are configured as two interferometers.

The output of the fiber coupler 505 is connected to one of the fiber cores 512 in the fiber bundle 508. The light from the fiber core 512 is focused on a sample 511 through a lens 509 and a scanning mirror 510. The backscattered photons from the sample 511 can be collected from the illuminated spot through the fiber core 512 shown as the solid line or from an offset position through another fiber core 513. The backscattered light out of the fiber core 512 interferes with the light out of the fiber coupler 506 in the fiber coupler 505. Similarly, the backscattered photons from the offset position collected by the fiber core 513 will be delivered to the fiber coupler 507 and interfere with the light from another output of the fiber coupler 506. The interference will be detected by the components 501 and 502, which may be a spectrometer (if the light source 503 is a broadband light source) or a photodetector (if the light source 503 is a swept light source). The light out of the fiber coupler 506 serves as reference arms for the interferometers built on fiber couplers 505 and 507. The optical path length of the reference arms must match the length of the sample arm constructed by the fiber core 512 or 513 and lens 509 and scanning mirror 510.

In the fiber bundle 508, multiple fiber cores can be used to collect light from different offset positions as shown in the illustration of the cross section of the fiber bundle 508 having multiple cores 514. The invention only requires a lens 509 after the fiber bundle 508 for either collimation or focusing. The light should not be collimated from each core. To scan a cavity, the mirror 510 can be rotated to form circumferential scanning or the fiber bundle can be vibrated without the mirror 510 to form forward scanning.

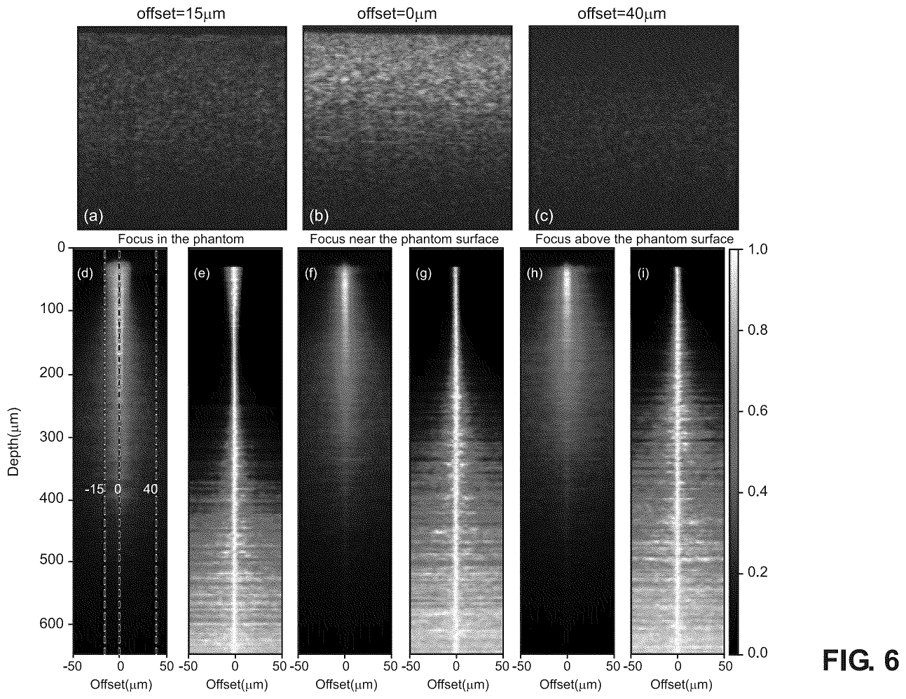

As an experiment, a solid phantom was constructed by mixing 2% agarose with 0.5% intralipid and then was imaged by the FIG. 8 apparatus. The total offset is ±50 μm. FIG. 6 shows a Backscattered Photon Profile (BSPP) with different focal positions in the phantoms. FIGS. 6(a)-(c) show three FSOCT A-scan images acquired by the FIG. 8 embodiment when the offsets of the focus on the surface of the phantom were at −15 μm (FIG. 6(a)), 0 μm (FIG. 6(b)), and +40 μm (FIG. 6(c)). That is, the A-scan images in FIGS. 6(a)-(c) are representative FSOCT images acquired at −15 μm (which is a distance 15 μm from the illumination point), 0 μm (which is at the illumination point), and 40 μm (which is a distance 40 μm from the illumination point), respectively.

For the OCT image at each position, a mean A-scan was calculated from all A-scans (to suppress the speckles). It is known in the art that each A-scan contains numerical data related to photons that were detected, and data relates to the depth of the photons. Further mean and average are examples of mathematical processes by which speckles may be mitigated or eliminated. The BSPP was then reconstructed in a logarithmic scale shown in FIG. 6(d) using the mean A-scan against the offset. Using the same dataset as FIG. 6(d), the intensity at each depth was normalized and the BSPP was plotted on a linear scale shown in FIG. 6(e) as predicted in Eq. (3). Thus, the images of FIGS. 6(d) and (e) are reconstructed BSPPs in logarithmic scale and normalized linear scale, respectively, with the focus within the intralipid phantom. The three dashed green lines in FIG. 6(d) indicate the offset positions of the images of FIG. 6(a)-(c).

To further validate observations, the focus was shifted to a location at the phantom surface (as shown in FIGS. 6(f) and (g)). The focus was also shifted to be above the phantom surface (as shown in FIGS. 6(h) and (i)). The images in FIGS. 6(g) and (i) show the corresponding shift of the focus to the expected position at and above the phantom surface, respectively. The images of FIGS. 6(f) and (g) are reconstructed BSPPs in logarithmic scale and normalized linear scale, respectively, with the focus on the surface of the intralipid phantom. The images of FIGS. 6(h) and (i) are reconstructed BSPPs in logarithmic scale and normalized linear scale, respectively, with the focus above the phantom surface.

With the data and images described above, one can observe how the illumination beam is focused and spread out in the scattering medium because the profile of the beams as shown in FIGS. 6(e), (g) and (i) simulated the shape of the beam in the medium. The BSPP can be approximated as a Gaussian beam, especially around the focus, as derived in Eq. (5). This allows the location of the focus in the medium to be determined mathematically, by viewing or processing the images and in other ways apparent to persons of ordinary skill from this description.

Therefore, it is possible to determine the focal point in a medium by taking OCT images at various offset positions, calculating the average or mean A-scan by taking the mean of all the data from the A-scans at that location and then reconstructing the BSPP using the mean A-scan against the offset. The BSPP may be displayed on a logarithmic scale, and using the same dataset the intensity at each depth and plotted the BSPP may be normalized on a linear scale as predicted in Eq. (3). The focal point is the point on the image (and in the normalized data) where the illumination beam is narrowest. The process displays the beam profile at various positions as a function of the OCT images, which permits a calculation of where the beam is narrowest, thereby permitting the determination of where the focus is located in the medium. This gives information about how the beam is distributed inside of the human tissue. It has not been previously possible to obtain this type of information about how the beam is focused and distributed in tissue.

There is a desire to separate least scattering photons (LSPs) from multiple scattering photons (MSPs). As shown in FIG. 6, the BSPP consists of LSPs as the central beam and MSPs as the skirt. A two-Gaussian function may be used to fit the LSPs central beam and the MSPs skirt to separate them.

Another function that corresponds to PSF is called Modulation Transfer Function (MTF). MTF is calculated from the Fourier transform of the PSF correlation function represented by BSPP, and it is another way to characterize an optics system. A depth-resolved MTF can therefore be obtained.

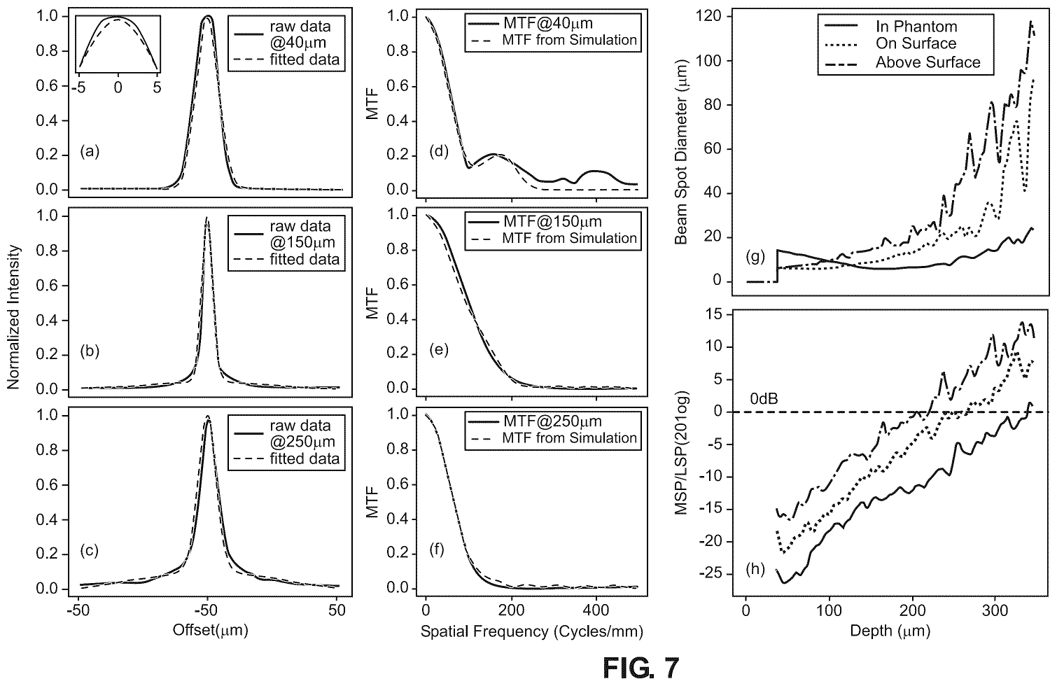

FIG. 7 shows the quantification of MTFs, beam waist, and contrast. FIGS. 7(a)-(c) are three representative PSF autocorrelations showing the measured BSPPs and fitted curves at three different depths. Because the central beam, made up of LSPs, can be written as the PSF autocorrelation based on Eq. (3), one can calculate the Fourier transform of the autocorrelation function to access the MTFs at different depths. The measured MTFs and simulated MTFs may be plotted in optical design software in FIGS. 7(d)-(f). The calculated MTFs from the FSOCT are in good agreement with the simulated results, indicating that FSOCT can access depth-resolved MTF using the LSPs central beam.

The inset in FIG. 7(a) shows an apparent deviation between the raw data and the fitted data at the top of the PSF autocorrelation at 40 μm. The deviation indicates that the illumination beam at this depth does not resemble a Gaussian function. At the other two depths, MSPs skirts are shown at the bottom of PSFs.

FIG. 7(g) shows the beam waist change along with the imaging depth using the data in FIGS. 6(e), 6(g), and 6(i). With the fitted two-Gaussian function, the beam waist variation can be quantified along with the imaging depth. The beam waists at 1/e² can be calculated using the two-Gaussian function fitted data.

FIG. 7(h) shows the distribution of MSPs and LSPs at different depths calculated as the ratio of MSPs to LSPs from the data in FIG. 1. FIG. 7(h) was quantified from the data of FIGS. 6(e), 6(g), and 6(i). The 0 dB dash line in FIG. 7(h) indicates the critical imaging depths.

With the flow chart of FIG. 12 as a guide, one may see how the equipment and methods described above may be utilized. As a first step in the flow chart of FIG. 12, images are obtained using FSOCT and FSOCM devices. With the equipment described and shown above, for example as shown in FIGS. 8, 10, 10B, 11 and 11B, OCT images at the illumination point and at offset positions can be captured simultaneously. Once this is accomplished, one may move to step two, which is to reconstruct a Back Scattering Projection Pattern (BSPP). To reconstruct a smooth BSPP, speckles at each offset position can be suppressed by acquiring an OCT image across a small range at each offset position, for example, 50 μm. The image at an offset position may look like the images shown in FIGS. 6(a)-(c). Then all A-scans in an OCT image can be averaged to a signal shown in BSPP at the corresponding offset position. For example, the green dashed lines in FIG. 6(d) show the positions of the averaged offset OCT signals from the FIGS. 6(a)-(c).

The present disclosure contemplates applying standard OCT or OCM signal processing to each detector on the detector array. The reconstructed BSPP can be displayed in a two dimensional image, similar to that shown in FIG. 6, or in a three dimensional image, or as an enface view at a specific depth, for example with OCM. In OCT, multiple A-scans are acquired as the scanner scans across the sample to form a B-scan or even a C-scan. In FSOCT, the images are acquired simultaneously at the illumination point and at offset points. To suppress the speckles of the BSPP, neighbor A-scans acquired by the FSOCT can be averaged to generate a smooth BSPP, as shown in FIG. 6. The neighbor or offset A-scans are the data acquired by the detector that are close to the A-scan at the illumination spot.

From the BSPP, one can carry out one or more of at least three other steps or methods (see FIG. 12 top right three boxes), which are Focus Tracking, Extracting of Tissue Optical Properties, and Feedback for Adaptive Imaging. Alternatively, one may recover the phases based on parallel detection, which gains sufficient information to enable carrying out Complex Variation, as noted in FIG. 12 without the BSPP reconstruction. Recovery of the phases is described immediately below.

In conventional OCT, the phase of the OCT signal is not stable during mechanical scanning. This is because small amounts of motion by the subject can create large amounts of noise, preventing accurate extraction of the phase information. However, with the FSOCT/FSOCM methods carried out as described above using the devices disclosed above, the OCT signals from the illumination spot and the surrounding spots are acquired simultaneously. This simultaneous acquisition of OCT images guarantees a stable phase because there can be no motion between the acquisition of the images. This stability in the phase permits the phase information to be extracted through the inverse Fourier transform of the image at an imaged location. From another point of view, FSOCT/FSOCM can be considered the diffraction pattern of an imaged subject.

Complex variation: Complex OCT signal can be written as S_{oct}=Ae^{iψ}. By exploring the phase variation (iψ), OCT can be used to extract flow or tiny motion in a subject, for example, the blood cell motion in blood vessels. Conventional OCT compares the difference between two OCT A-scans at different time points T1 and T2 (see FIG. 15) as

$$D = \frac{{A}_{1} {e}^{i ( {ψ}_{1 +} {ψ}_{n o i s e 1} )}}{{A}_{2} {e}^{i ( {ψ}_{2} + {ψ}_{n o i s e 2} )}} = \frac{{A}_{1}}{{A}_{2}} {e}^{i [ ( {ψ}_{1} - {ψ}_{2} ) + ( {ψ}_{n o i s e 1} - {ψ}_{n o i s e 2} ) ]}$$

However, the noise associated with the phase at different times T1 and T2 could be different. Although phase differences are very sensitive to motion, they are overshadowed by noise. In other words, it may be difficult to differentiate the phase variations induced by motion and noise. With FSOCT/FSOCM, the influence of the noise on the phase variation induced by motion is eliminated when phases at different offset positions are acquired parallelly at the same time. As shown in FIG. 15, at a specific time T1, the OCT signals between two offset positions can be divided as

$${S}_{1 offset\_D} = \frac{{A}_{1} {e}^{i ( {ψ}_{1 +} {ψ}_{n o i s e 1} )}}{{A}_{2} {e}^{i ( {ψ}_{1 off} + {ψ}_{n o i s e 1} )}} = \frac{{A}_{1}}{{A}_{2}} {e}^{i ( {ψ}_{1 -} {ψ}_{1 off} )}$$

When imaging a flow with scattering particles, such as red blood cells at a specific time T1, the FSOCT phase signals between two offset positions can be also directly written as the following equation to get the phase difference.

Ψ_{1D}=(ψ₁₊ψₙₒᵢₛₑ₁)−(ψ_{1off}+ψₙₒᵢₛₑ₁)=(ψ₁−ψ_{1off})

The phase noise term is eliminated due to both FSOCT signals being acquired at the same time. When the noise has been removed from the above operation, then one can compare the difference between two FSOCT signals at two time points T1 and T2 as

S_{D}=S_{1offset_D}−S_{2offset}_{_{D}} or Ψ_{D}=Ψ_{1D}−Ψ_{2D}=(Ψ₁−Ψ₂)+*Ψ_{2off}−Ψ_{1off})

It should be noted that phase variation is different at different locations. For example, if (Ψ_{2off}−Ψ_{1off}) is acquired for a region that does not have a flow or the flow rate is small, then (Ψ_{2off}−Ψ_{1off})≈0. One can extract the absolute phase variation only due to the flow as(Ψ₁−Ψ₂) without the influence of noise. For the purpose of illustration, only two positions are shown and described. In practice, the FSOCT signal from multiple offset locations can be processed similarly. As the noise has been removed, the signal-to-noise ratio can be significantly improved.

After the FSOCT or FSOCM images have been acquired, the BSPP may be reconstructed and the phase information is recovered, the imaging methods for different applications may be carried out as noted in the far right boxes of FIG. 12.

In accordance with equations (4) and (5) and FIG. 6, the BSPP can provide valuable information on the illumination beam distribution within a scattering medium, including the focal position, depth, and beam waist. In various applications, tracking the focus of the illumination beam is crucial. As illustrated in FIG. 14, the FSOCT illumination beam 301 can be focused on the tissue 304 through a lens 303. Using the BSPP, the focal position can be identified on the component 305. To maintain or lock the focal position in the tissue, an optical focusing power-adjustable component 302, such as a phase modulator, a variable focus liquid lens or a scanner, can be utilized to adjust the focus in the tissue 304 based on the focal position shown in the BSPP.

By compensating for variations in the focal position, accurate tissue structure quantification can be achieved, which is essential for monitoring disease progression over extended periods. In addition to controlling the focus, the recorded focal position during the image can also be used to remove motion artifacts. One method of controlling the focus is to obtain OCT images and focus on a particular depth of tissue, such as the human retina. However, it is not clear with conventional OCT that the focus of the beam is at a particular depth of the tissue. With FSOCT, the focal point, in terms of depth in the tissue, can be tracked with the methods and devices described herein. If the focal point can be tracked, then even if a patient moves slightly the focal point can be moved to stay at the same depth in the tissue or the focus can be locked on a specific feature similar to focus-locking during photography.

Feedback for adaptive imaging: In tissue imaging, such as the retina, the wavefront of the illumination beam can be distorted by the aberrations, such as those induced by the cornea and lens. This results in a reduction of the lateral resolution of images. Adaptive imaging can address this issue by using a wavefront shaping component to compensate for the distortion and focus the beam to a diffraction-limited spot on the targeted tissue. This can be achieved by obtaining the distorted wavefront of the illumination beam prior to compensation or by using a metric as an indicator during optimization. Various methods, such as OCT, confocal, and nonlinear imaging, have been developed for adaptive imaging. However, these methods still have limitations, such as complexity, cost, or phototoxicity.

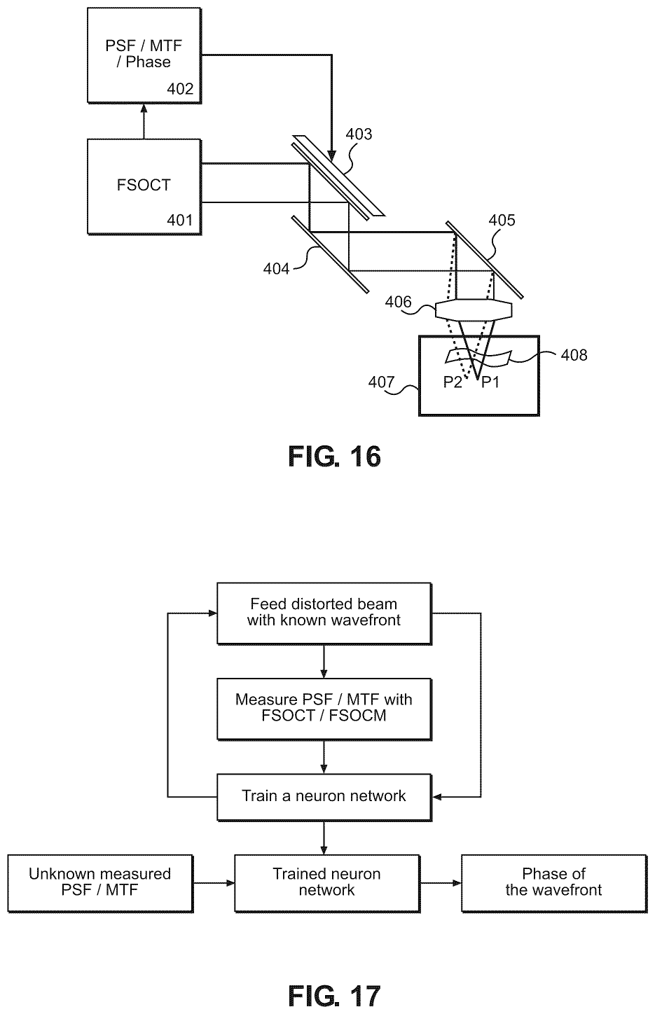

Adaptive imaging by optimizing PSF/MTF. Because PSF/MTF can be accessed through FSOCT/FSOCM, PSF/MTF may be used as the metric to realize adaptive imaging. FSOCT/FSOCM can obtain depth-resolved PSF/MTF in scattering media. In FIG. 16, based on measured PSF/MTF 402, the wavefront shaping component 403, such as a deformable mirror, can be employed to modify the wavefront of the illumination beam, which is focused onto a sample 407 through a lens/lenses 406. The wavefront shaping component 403 can shape the wavefront of the illumination beam until the best PSF/MTF of the imaging system is reached. Usually, the narrowest BSPP or smooth and broad MTF indicates that the system has been optimized close to diffraction-limited performance. Such optimization may be done through a number of iterations.

Adaptive imaging through neuron network training. In one embodiment, a method for utilizing a deep-learning neural network to extract the phase of a wavefront is disclosed. The method comprises the steps of providing a series of light beams with known wavefront distortion, measuring the Point Spread Function (PSF) or Modulation Transfer Function (MTF) using FSOCT or FSOCM, and training the neural network using the measured PSF or MTF. This will proceed for many different light beams with known wavefront distortions and measuring the PSF or MTF. Once this has occurred, this trained neural network will be used to derive the phase of an unknown distorted wavefront when the PSF or MTF is known. The wavefront shaping component then generates the opposite of the phase of the distorted wavefront to correct the distorted wavefront at the focal spot. A flow chart demonstrating the method is depicted in FIG. 17.

Adaptive imaging through extracting phase variation for OCT complex signal. The wavefront of a light beam is determined by the phase of a light wave. If the phase of the light beam can be directly extracted (measured), then the opposite phase of the distorted wavefront can be provided to the wavefront shaping component 3 to compensate for the distortion without requiring iteration. This method includes the determination of the phase of the wavefront by eliminating noise, and avoids the need to either perform numerous iterations or train a neural network, as the previous two methods describe. The illustration of the method can be shown to be used with the FIG. 16 equipment.

FSOCT/FSOCM parallelly captures the complex OCT signal as S1 (x1, y1, z) at P1 and S2 (x2, y2, z) at P2, as shown in sample 407 of FIG. 16. P1 and P2 represent two different imaged locations, which are physically close but generate random and independent OCT signals. These signals can be obtained by scanning the illumination beam a small distance (e.g., 5 microns) across the imaged subject to gather signals at numerous locations. The phase of each of the signals collected includes two parts: (1) the aberrated wavefront due to refractive index variation 408 (ψr), such as induced by the lens or the cornea, and (2) random phase variation (ψs), due to scattering, which is shown as speckles in OCT images. Such phase signal S can be obtained from obtaining OCT signals at many locations, and can be directly extracted after scanning and averaging as

$$ψ = \frac{[ ( {ψ}_{r} + {ψ}_{s 1} ) + ( {ψ}_{r} + {ψ}_{s 2} ) + … + ( {ψ}_{r} + {ψ}_{s n} ) ]}{n} = {ψ}_{r}$$

This results in the phase signal S because from different imaged locations, the aberrated wavefront ψr is similar, but random phase variation ψs is random. After averaging a large number (e.g., about 100 or more) of such measurements as shown in the equation above, only the aberrated wavefront ψr will remain because ψs is cancelled by the averaging calculation due to its randomness. Once the aberrated wavefront is obtained, the wavefront shaping component 3 can generate −ψr (“negative ψr”, which is a shape that is the opposite of ψr), which compensates for the induced wavefront distortion. Even though the signals are measured at different times and different locations, this is acceptable because the distortion (or aberration) does not change substantially over time and location.

Extracting tissue optical properties. Tissue optical scattering coefficient, absorption coefficient, and anisotropy (g) are valuable for diagnosis. Although various methods have been proposed to estimate the optical properties based on OCT images, the estimation requires prior knowledge of the optical systems such as wavelength, focal location, and refractive index of the imaged subject. And almost all models ignore MSPs by considering only LSPs. It remains challenging to translate the technology into clinics.

With FSOCT/FSOCM, LSPs and MSPs can be separated using function fitting. To separate LSPs and MSPs, the BSPP is first normalized. This was described above in relation to FIG. 6 at each depth and then fitted with functions, such as a two-Gaussian function as

G(Δr)=G_{L}(Δr)+G_{M}(Δr)

Here, G_{L}(Δr) was used to fit the LSPs central beam and G_{M}(Δr) was used to fit the MSPs skirt. Both are Gaussian functions as

$${G}_{L} ( Δ r ) = C {e}^{- \frac{Δ {r}^{2}}{{w}_{L}^{2}}} and {G}_{M} ( Δ r ) = ( 1 - C ) {e}^{- \frac{Δ {r}^{2}}{{w}_{M}^{2}}}$$

Here, C and (1-C) are the coefficients of G_{L}(Δr) and G_{M}(Δr), w_{L} is the beam waist of the LSPs central beam and wM is the beam waist of the MSPs skirt. Here, we use Gaussian function as the example. Other functions, such as Lorenz function, can also be used.

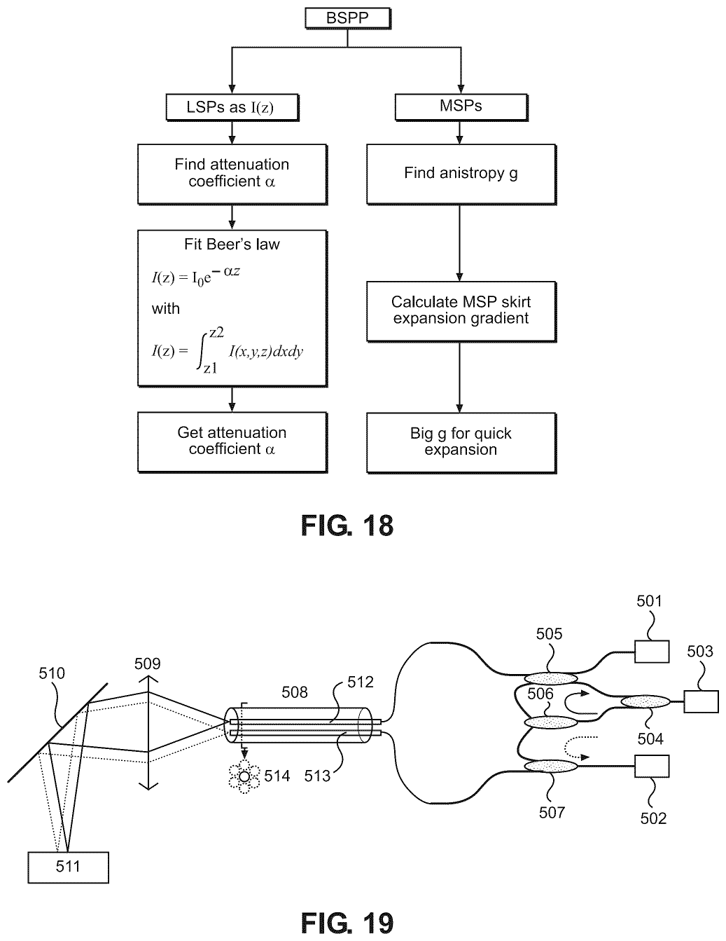

FIG. 18 shows the flow chart of extracting the attenuation coefficient and anisotropy. The BSPP is separated to LSPs central beam and MSPs skirt. The LSPs central beam can be used to fit Beer's law to extract the attenuation coefficient. The expansion of the MSPs skirt is related to anisotropy g. The MSP skirt expansion gradient (SEG) in terms of depth can be calculated to find out g at different depths. The gradient can be calculated as

$$SEG = \frac{The width of the MSP skirt at depth 1}{The width of the MSP skirt at depth 2}$$

Objectively quantify stray light in eye: As depth-resolved PSF and MTF can be accessed and LSPs and MSPs can be separated, this technology can be used to quantify the ocular stray light, such as the stray light induced by a cataract. One can capture the PSF from the top surface of the retina. By quantifying the contribution of MSPs through MSPs skirt, the stray light induced by the crystal lens can be evaluated. A method of capturing backscattered photons from a position that has a small offset from the illuminated area.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. An improved interferometer having a broadband light source, at least one beam splitter configured to split the broadband light source into at least a reference beam and a sample beam that is projected onto a sample and reflected back to the at least one beam splitter, the improvement comprising:
(a) a first detector array configured to receive a first portion of the sample beam light reflected back to the at least one beam splitter, wherein the first detector array includes a first plurality of photodetectors; and
(b) a first spatial filter at a first orientation relative to the sample, wherein the first spatial filter is positioned between the at least one beam splitter and the first detector array and configured to disperse the first portion of the sample beam light.

2. The improved interferometer in accordance with claim 1, further comprising:
(a) a second detector array configured to receive a second portion of the sample beam light reflected back to the at least one beam splitter, wherein the second detector array includes a second plurality of photodetectors; and
(b) a second spatial filter at a second orientation relative to the sample, wherein the second spatial filter is positioned between the at least one beam splitter and the second detector array and configured to disperse the second portion of the sample beam light;
wherein the second orientation is transverse to first orientation, and the second portion of the sample beam light is different from the first portion of the sample beam light.

3. The improved interferometer in accordance with claim 1, further comprising a dispersive component positioned between the first spatial filter and the first detector array for dispersing the light reflected back to the at least one beam splitter onto the first detector array.

4. The improved interferometer in accordance with claim 1, further comprising an adjustable focus.

5. The improved interferometer in accordance with claim 1, further comprising a scanner formed with a grouping of single-mode fibers, which scanner is configured to deliver the sample beam through at least one of the single-mode fibers and collect light reflected back from offset positions through multiple single-mode fibers.

6. An improved interferometer having a swept light source, at least one beam splitter that is configured to split the light source into at least a reference beam and a sample beam that is projected onto a sample and reflected back to the at least one beam splitter, the improvement comprising:
(a) a detector array configured to receive at least a portion of the light reflected back to the at least one beam splitter, wherein the detector array includes a plurality of photodetectors; and
(b) a lens positioned between the at least one beam splitter and the detector array that is configured for focusing at least a portion of the light reflected back to the at least one beam splitter onto more than one of the plurality of the photodetectors on the detector array.

7. The improved interferometer in accordance with claim 6, further comprising a scanner formed with a grouping of single-mode fibers, which scanner is configured to deliver the sample beam through at least one of the single-mode fibers and collect light reflected back from offset positions through multiple single-mode fibers.

8. An improved interferometer having a broadband light source, at least one beam splitter that is configured to split the light source into at least a reference beam and a sample beam that is projected onto a sample and reflected back to the at least one beam splitter, the improvement comprising:
(a) a detector array configured to receive at least a portion of the light reflected back to the at least one beam splitter, wherein the detector array includes a plurality of photodetectors;
(b) a lens positioned between the at least one beam splitter and the detector array that is configured for focusing at least a portion of the light reflected back to the at least one beam splitter onto more than one of the plurality of the photodetectors on the detector array;
(c) a phase modulator for introducing phase modulation; and
(d) a demodulator for extracting enface view images at specific depths based on the phase modulation.

9. The improved interferometer in accordance with claim 8, further comprising a scanner formed with a grouping of single-mode fibers, which scanner is configured to deliver the sample beam through at least one of the single-mode fibers and collect light reflected back from offset positions through multiple single-mode fibers.

10. A method of reconstructing a backscattered photon profile (BSPP) in a scattering medium, the method comprising:
(a) acquiring a B-scan image by scanning an illuminated point and multiple offset positions;
(b) calculating an average A-scan from the plurality of A-scans in the B-scan at each offset position; and
(c) constructing a BSPP against the offset positions.

11. The method in accordance with claim 10, further comprising adjusting an adjustable focus as a function of the focal point of the light source.

12. The method in accordance with claim 10, further comprising determining a location of one of the offset positions by a row or a column of a CCD the light falls onto.

13. A method of recovering depth-resolved PSF and MTF from a backscattered photon profile (BSPP):
(a) acquiring a first BSPP using FSOCT or FSOCM with apertures of a predetermined size for an illumination beam and a detection beam to neglect aberration effects;
(b) increasing the size of at least one aperture for the illumination beam or the detection beam in order to acquire a second BSPP, thereby introducing aberration effects;
(c) deconvolving the second BSPP using the first BSPP to obtain a depth-resolved Point Spread Function (PSF);
(d) conducting Fourier transform on the depth-resolved PSF to obtain a depth-resolved MTF; and
(e) utilizing the obtained depth-resolved MTF for diagnostic or imaging purposes.

14. The method in accordance with claim 12, wherein the deconvolution of the second BSPP is conducted mathematically, such as by Fourier analysis, deconvolution algorithms, and other signal processing methods.

15. The method in accordance with claim 12, wherein the step of utilizing the obtained depth-resolved MTF further comprises identifying aberrations or evaluating image quality.

16. A method for maintaining a focal position of an imaged subject, the method comprising:
(a) utilizing BSPP or depth-resolved MTF to identify a first focal position;
(b) using the first focal position as feedback to adjust a focal length or a distance between a lens and the imaged subject; and
(c) locking the focal position of the imaged subject by maintaining the adjusted focal length or the distance between the lens and the imaged subject.

17. A method of extracting phase variation induced by moving subjects, the method comprising:
(a) acquiring simultaneously first and second OCT signals at offset positions using an FSOCT or FSOCM process;
(b) separating the acquired first and second OCT signals into amplitude and phase components using mathematical techniques;
(c) acquiring third and fourth OCT signals at the same position and different time points using an FSOCT or FSOCM process;
(d) subtracting a phase signal between the first and second FSOCT/FSOCM signals to remove phase noise;
(e) subtracting a phase signal between the third and fourth FSOCT/FSOCM signals; and
(f) analyzing the resulting signal to represent the moving subjects.

18. The method in accordance with claim 17, further comprising subtracting the results of steps (d) and (e) from one another to remove phase noise.

19. The method in accordance with claim 17, wherein the mathematical techniques for separating the OCT signals into amplitude and phase components comprise Fourier analysis, Hilbert transform and other signal processing methods.

20. A method for adaptive imaging comprising:
(a) deriving or measuring a distorted wavefront of an illumination beam due to aberration;
(b) generating an opposite wavefront to compensate for the distortion through a wavefront shaping device; and
(c) using an FSOCT or FSOCM process to derive or measure the illumination beam wavefront.

21. The method in accordance with claim 20, wherein the step of deriving the illumination beam wavefront comprises:
(a) using PSF/MTF as the metric to derive the illumination beam wavefront; and
(b) monitoring continually the PSF/MTF until it reaches a diffraction limit of the imaging system through iteration.

22. The method in accordance with claim 20, wherein the step of deriving the illumination beam wavefront comprises:
(a) inputting known distorted wavefronts into a neuron network;
(b) measuring PSF/MTF of the known distorted wavefronts with BSPP;
(c) training the neuron network with known distorted wavefronts and measuring PSF/MTF derived from the BSPP; and
(d) inputting a measured PSF/MTF to the trained neuron network to derive the distorted wavefront.

23. The method in accordance with claim 20, wherein the step of deriving the illumination beam wavefront comprises:
(a) scanning the beam across a small range;
(b) extracting the phase terms from complex OCT signals at all offset positions;
(c) averaging the phase terms at each offset position using the OCT signal at all locations in the scanning range; and
(d) using the averaged phase terms at different offset positions as the wavefront of the illumination beam at the focal position.

24. A method of separating least scattered photons (LSPs) from multiple scattered photons (MSPs) in tissue imaging, the method comprising:
(a) using a mathematical function to fit BSPP data;
(b) attributing a first of the mathematical functions to the central bright beam dominated by LSPs; and
(c) attributing to a second of the mathematical functions to the skirt beam dominated by MSPs.

25. The method in accordance with claim 24, wherein the mathematical function is a two-Gaussian function.

26. The method in accordance with claim 25, wherein the central bright beam is used to fit Beer's law equation to extract the attenuation coefficient of the imaged tissue.

27. The method in accordance with claim 24, wherein an expansion gradient of the skirt beam dominated by MSPs represents the anisotropy of the imaged subject in which higher expanding gradient indicates a larger anisotropy coefficient of the tissue.
